(19) United States
(12) Patent Application Publication
(10) Pub. No.: US 2008/0206363 A1
(43) Pub. Date: Aug. 28, 2008

(54) Skin stabilizer preparation

(76) Inventor: Steve Harlan, Des Moines, IA (US)

Correspondence Address: ROTHWELL, FIGG, ERNST & MANBECK, P.C., 1425 K STREET, N.W., SUITE 800, WASHINGTON, DC, 20005, US

(21) Appl. No.: 11/711,640

(22) Filed: Feb. 28, 2007

(51) Int. Cl. A61K 33/04

(52) U.S. Cl. 424/713

(57) ABSTRACT

A method for managing the tolerability of a patient to treatment with a drug that may cause dermatological side effects is provided. An effective amount of a composition which comprises a corticosteroid and sulfur is administered to the skin of such patient.

### CROSS REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

The present invention generally relates to a skin stabilizer preparation to aid in the treatment and prevention of various skin conditions. More particularly, the present invention relates to a skin stabilizer preparation containing a corticosteroid and sulfur.

2. Description of the Related Art

Some skin conditions are side effects of strong topical medications used to treat other adverse skin conditions, for example, irritation from topical 5-FU treatment of actinic keratoses on the face and arms, and irritation from retinoids. Often patients with very sensitive skin cannot tolerate commonly used topical medications such as Benzamycin gel (trademark), retinoids (such as tretinoin, tazarotene and adapalene), benzoyl peroxide products, metronidazole preparations, and alpha hydroxyl acid preparations. Therefore, there is a need for a preparation that stabilizes the skin for other skin care medications.

### BRIEF SUMMARY OF THE INVENTION

In one aspect, the present invention provides a method for managing the tolerability of a patient to treatment with a drug that may cause dermatological side effects, said method comprising administering to the skin of said patient an effective amount of a composition which comprises a corticosteroid and sulfur.

In another aspect, the present invention provides a composition for managing the tolerability of a patient to treatment with a drug that may cause dermatological side effects, said composition comprising an effective amount of a corticosteroid in admixture with an effective amount of sulfur.

### DETAILED DESCRIPTION OF THE INVENTION

As noted above, the use of strong topical medications used to treat adverse skin conditions can cause its own dermatological side effects. For example, topical treatment of actinic keratoses on the face and arms with 5-FU, or treatment of various skin conditions with retinoids, can lead to significant skin irritation. Often patients with very sensitive skin cannot tolerate commonly used topical medications such as Benzamycin gel (trademark), retinoids (such as tretinoin, tazarotene and adapalene), benzoyl peroxide products, metronidazole preparations, and alpha hydroxyl acid preparations. It has been discovered that such side effects can be reduced by the topical application of the compositions of the present invention, thus effectively increasing the patient's tolerance of the underlying drug treatment.

The present compositions and methods may be used in conjunction with any dermatological therapy that may result in adverse side effects that affect the skin. In a preferred example, the present compositions and methods are useful in reducing skin irritation caused by retinoid therapy, especially therapy with tretinoin.

An important aspect of the preparations of the present invention is that they contain a combination of a corticosteroid and sulfur. In a preferred embodiment, the preparation also contains instructional material for use.

The corticosteroid component is preferably prescription strength, although a non-prescription strength corticosteroid composition is still contemplated in the invention. The corticosteroid is preferably present in the composition in an amount of from about 0.01% to about 2.0% (weight/weight (“w/w”)), more preferably about 0.75% (w/w).

The sulfur is preferably present in an amount of from about 0.25% to about 2.0%, more preferably about 0.5%.

Many corticosteroids are suitable for use in connection with the present invention, including, for example, hydrocortisone, hydrocortisone acetate, 0.05% triamcinolone, 0.075% betamethasone dipropionate, and 0.04% clobetasol. The sulfur component may be any suitable form, including precipitated sulfur.

The present compositions containing the corticosteroid and the sulfur may include one or more conventional pharmaceutically acceptable carrier components to form suitable topical formulations. A wide range of formulations are contemplated, for example ointments, lotions, creams, gels, drops, sprays, and liquids. Lotions are particularly preferred.

When the preparations of the invention are used to manage drug tolerability, the preparation may be applied to the skin either before, after, or at essentially the same time as the drug. It is contemplated that the user would apply the preparation to necessary areas of the skin at least once daily, preferably two times daily.

The following is a non-limiting example of a hydrocortisone composition for the skin stabilizer preparation.

### EXAMPLE 1

### Formula for Dermatologic Lotion for Managing Retinoid Tolerability

### Phase 1:

| Water | qs 100% w/w |
|---|---|
| Potassium sorbate | 0.2 |
| Carbomer 934 | 0.3 |

### Phase 2:

| | |
|---|---|
| Glycerin | 3.0 |
| Magnesium | 1.0 |
| Aluminum Silicate | |

### Phase 3:

| | |
|---|---|
| Petrolatum | 6.0 |
| Dimethicone | 3.0 |
| Steareth-2 | 3.0 |
| Laureth-23 | 1.0 |
| Cetyl alcohol | 1.0 |

### Phase 4:

| | |
|---|---|
| Water | 2.0 |
| Glycerin | 2.0 |
| Hydrocortisone USP | 0.25–2.0 |
| Sulfur | 0.25–2.5 |

### Phase 5:

| Water | |
|---|---|
| Benzyl alcohol | 1.0 |
| Methylparaben | 0.2 |
| Propylparaben | 0.15 |
| Sodium Benzoate | 0.1 |
| Trisodium EDTA | 0.1 |

### Phase 6:

| | |
|---|---|
| Sodium hydroxide, 10% sol. | qs pH ~6.5–7.0 |

Compounding procedure:
1. Disperse Carbomer in Phase 1 water.
2. Disperse Mg Al Silicate in Phase 2 Glycerin.
3. Add Phase 2 to Phase 1 and heat with non-vortex mixing to 70 degrees C.
4. Combine Phase 3 ingredients and heat to 72 degrees C.
5. Add Phase 3 to Phase 1+2 to form emulsion.
6. Combine Phase 4 ingredients to form a slurry. Add to emulsion when its temperature has dropped to 55 degrees C.
7. Homogenize emulsion to completely disperse actives.
8. Combine Phase 5 ingredients and add to emulsion when its temperature has dropped to 45 degrees C.
9. Adjust emulsion pH to 6.5-7.0 using NaOH 10% aqueous solution once its temperature has dropped to 40 degrees C.
10. As emulsion thickens, change from propellor to sidesweep mixing and continue cooling to ambient temperature.

The stabilizer prevents and treats irritation from strong prescription topical medications. Patients who are unable to tolerate Benzamycin gel (trademark) therapy for acne therapy can continue on their prescriptions of Benzamycin gel when they use a small amount of the skin stabilizer preparation once or twice daily. This also true for patients with sensitive skin and/or “combination skin” who have been prescribed retinoid products, and benzoyl peroxide products including the Benza-products for acne, the Metronidazole products for rosacea, and topical 5-FU products for actinic keratoses.

The inclusion of elemental sulfur (PS) in the skin stabilizer preparation inhibits the overgrowth of Pityrosporum yeast and bacteria in the skin. This allows relatively lower dosages of corticosteroid in the stabilizer system to be used safely for an extended period of time (almost indefinetly). In a clinical review of 330 patients with long term use, there were no incidents of steroid dermatitis or steroid side effect. Therefore, people with some chronic skin conditions or a skin condition that takes a very long time to treat or stabilize, for example, eczema, psoriasis, people with “combination skin,” red bumpy skin around the nose and mouth, mild acne, shaving rashes, redness from rosacea, and sun damaged red rough skin, will consistently benefit from the skin stabilizer. The stabilizer does not contain sulfacetamide and can be used by patients allergic to the sulfa anti-biotics, but can contain safe natural ingredients including fragrance. Further, a preferred embodiment of the current invention does not contain a medication which often causes skin irritation (e.g. salicylic acid) to maximize the stabilization effect of the preparation.

Because the skin stabilizer preparation contains a lower and safer dose of corticosteroid and precipitated sulfur, it enables users to minimize adverse side effects from which users often suffer when they are exposed to a strong dose of corticosteroid or sulfur over an extended period of time. The stabilizer preparation of the invention is safer in terms of adverse effects, but often is just as effective as high dose or full dose of corticosteroid or sulfur for many users. The low doses of corticosteroid and sulfur produce efficacy, prevents development of resistance, and provides more safety from side effects.

While preferred embodiments of the present invention have been described above, it should be understood that they have been presented by way of example only, and not limitation. Thus, the breadth and scope of the present invention should not be limited by the above described exemplary embodiments.

Obviously, numerous modifications and variations of the present invention are possible in light of the above teachings. It is therefore to be understood that the invention may be practiced otherwise than as specifically described herein.

1. A method for managing the tolerability of a patient to treatment with a drug that may cause dermatological side effects, said method comprising administering to the skin of said patient an effective amount of a composition which comprises a corticosteroid and sulfur.

2. The method of claim 1, wherein the corticosteroid comprises hydrocortisone, triamcinolone, betamethasone dipropionate, or clobetasol.

3. The method of claim 2, wherein the corticosteroid comprises hydrocortisone acetate.

4. The method of claim 1, wherein the sulfur comprises precipitated sulfur.

5. The method of claim 1, wherein the concentration of the corticosteroid in the composition is from about 0.01% to about 2.0%.

6. The method of claim 5, wherein the concentration of the corticosteroid in the composition is about 0.75%.

7. The method of claim 6, wherein the concentration of the sulfur in the composition is from about 0.25% to about 2.0%.

8. The method of claim 7, wherein the concentration of the sulfur in the composition is about 0.5%.

9. The method of claim 1, wherein the drug treatment comprises treatment with a retinoid.

10. The method of claim 9, wherein the retinoid comprises tretinoin.

11. A composition for managing the tolerability of a patient to treatment with a drug that may cause dermatological side effects, said composition comprising an effective amount of a corticosteroid in admixture with an effective amount of sulfur, and a pharmaceutically acceptable carrier.

12. The composition of claim 11, wherein the corticosteroid comprises hydrocortisone, triamcinolone, betamethasone dipropionate, or clobetasol.

13. The composition of claim 12, wherein the corticosteroid comprises hydrocortisone acetate.

14. The composition of claim 11, wherein the sulfur comprises precipitated sulfur.

15. The composition of claim 11, wherein the concentration of the corticosteroid in the composition is from about 0.01% to about 2.0%.

16. The method of claim 15, wherein the concentration of the corticosteroid in the composition is about 0.75%.

17. The method of claim 16, wherein the concentration of the sulfur in the composition is from about 0.25% to about 2.0%.

18. The composition of claim 17, wherein the concentration of the sulfur in the composition is about 0.5%.

19. The composition of claim 11, which is in the form of an ointment, a lotion, a cream, a gel, a drop, a spray, or a liquid.

20. The composition of claim 19, which is in the form of a lotion.
